(19) United States
(12) Patent Application Publication
(10) Pub. No.: US 2025/0164505 A1
(43) Pub. Date: May 22, 2025

(54) DETECTION OF EXTRACELLULAR VESICLE BIOMARKERS FOR THE DIAGNOSIS OF AUTISM SPECTRUM DISORDER

(71) Applicant: Qatar Foundation for Education, Science and Community Development, Doha (QA)

(72) Inventors: Yongsoo Park, Doha (QA); Sara Abdulla, Doha (QA)

(21) Appl. No.: 18/629,294

(22) Filed: Apr. 8, 2024

Related U.S. Application Data
(60) Provisional application No. 63/601,041, filed on Nov. 20, 2023.

(51) Int. Cl. G01N 33/68

(52) U.S. Cl. CPC G01N 33/6893; G01N 2800/28

(57) ABSTRACT

Methods for early diagnosis of autism spectrum disorder, based on detecting certain protein biomarkers associated with plasma extracellular vesicles, are described.

### CROSS REFERENCE TO RELATED APPLICATIONS

This application claims the benefit of U.S. Provisional Application No. 63/601,041 filed on Nov. 20, 2023, which is incorporated herein by reference in its entirety.

### FIELD

This disclosure is related to testing for autism spectrum disorder.

### BACKGROUND

Diagnosing autism spectrum disorder (ASD) can be difficult because there is no medical test, like a blood test, to diagnose the disorder. Typically, doctors evaluate a child's developmental history and behavior to make a diagnosis. ASD can sometimes be detected at eighteen months of age or younger. By age two, a diagnosis by an experienced professional can be considered reliable. However, many children do not receive a final diagnosis until they are much older. Some people are not diagnosed until they are adolescents or adults. This delay means that people with ASD might not get the early help they need.

While the exact causes of ASD are not yet clear, both genetic and environmental factors might contribute to ASD etiology. Immune dysregulation, which represents the abnormal functioning of the immune system (e.g., chronic inflammation), is one of the environmental factors that can affect autism. The mechanisms by which immune dysregulation may influence ASD are not fully understood.

There is a need for tests that can diagnose ASD so that affected patients can get the help they need as early as possible.

### SUMMARY

Provided herein is a method for diagnosing autism spectrum disorder comprising
a) isolating extracellular vesicles (EVs) from plasma samples of subjects suffering from autism spectrum disorder; and
b) measuring protein expression levels in the extracellular vesicles.

### BRIEF DESCRIPTION OF THE DRAWINGS

FIG. 1 describes a schematic overview of the procedure for isolating extracellular vesicles (EVs) from plasma using size exclusion chromatography (SEC). EVs elute first followed by smaller protein complexes.

FIG. 2 shows a volcano plot of EV proteomes and demonstrates that certain proteins are downregulated in ASD. The differentially expressed (DE) proteins in plasma EVs isolated from healthy control (HC) and ASD cases; log₂ fold change (FC) against Limma −log₁₀ BH-adjusted p-value. Color indicates significantly downregulated (blue) proteins with FC≥2 and adjusted p-value<0.05.

FIG. 3 is a schematic overview of an enzyme linked immunosorbent assay (ELISA).

FIG. 4 shows 25 cytokines can be detected in plasma EVs using Bio-Rad Pro Human Cytokine screening panel, 48-plex.

FIG. 5 shows 6 cytokines are dysregulated in EVs isolated from ASD plasma, i.e., Gro-alpha, INF-gamma, IL-15, RANTES, SCGF-beta, and VEGF. The pro-inflammatory cytokine INF-Gamma is upregulated in ASD EVs.

### DETAILED DESCRIPTION

Extracellular vesicles (EVs) are membrane-bound vesicles that are secreted by cells and mediate intercellular communication by transferring biomolecules such as proteins. EVs can cross the blood-brain barrier (BBB) and also circulate in the blood, reflecting the state of the cells from which they originate and provide information on disease progression and severity. Therefore, the composition of plasma EVs can serve as a non-invasive source of biomarkers for neurological disorders.

Provided herein are certain biomarkers which enable early detection of ASD. Evaluation of the changes in the profiles of EV proteomes in ASD can facilitate the early diagnosis of ASD.

### Definitions

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

“WWP2” refers to WW domain-containing protein 2.

“CLEC1B” refers to C-Type Lectin Domain Family 1 Member B.

“HSP-27” refers to heat shock protein 27.

“CD40” refers to cluster of differentiation 40.

“FR-alpha” refers to folate receptor alpha.

“Gro-alpha” refers to chemokine growth-regulated protein alpha.

“INF-gamma” refers to interferon gamma.

“IL-15” refers to interleukin 15.

“RANTES” refers to a chemokine regulated upon activation, normal t cell expressed and presumably secreted.

SCGF-beta” refers to serum stem cell growth factor beta.

“VEGF” refers to vascular endothelial growth factor.

“Compared to control” refers to protein expression levels in samples obtained from healthy humans, i.e., humans not suffering from ASD.

As used herein, “protein expression levels” is used interchangeably with “biomarker expression levels.” “Abnormal” protein expression refers to increased protein expression or decreased protein expression compared to control.

Provided is a method for diagnosing autism spectrum disorder comprising:
a) isolating extracellular vesicles (EVs) from plasma samples of subjects suffering from autism spectrum disorder;
b) lysing or permeabilizing the EVs; and
c) measuring expression levels of proteins selected from WWP2, CLEC1B, HSP-27, CD40, and/or FR-alpha using an enzyme-linked immunosorbent assay (ELISA);
wherein increased expression of proteins selected from WWP2, CLEC1B, HSP-27, CD40, and/or FR-alpha compared to control is diagnostic of autism spectrum disorder.

In some embodiments, the method comprises measuring expression levels of at least one of WWP2, CLEC1B, HSP-27, CD40, and/or FR-alpha. In some embodiments, the method comprises measuring expression levels of at least two of WWP2, CLEC1B, HSP-27, CD40, and/or FR-alpha. In some embodiments, the method comprises measuring expression levels of at least three of WWP2, CLEC1B, HSP-27, CD40, and/or FR-alpha. In some embodiments, the method comprises measuring expression levels of at least four of WWP2, CLEC1B, HSP-27, CD40, and/or FR-alpha. In some embodiments, the method comprises measuring expression levels of WWP2, CLEC1B, HSP-27, CD40, and FR-alpha.

Provided is a method for diagnosing autism spectrum disorder comprising:
a) isolating extracellular vesicles (EVs) from plasma samples of subjects suffering from autism spectrum disorder;
b) lysing or permeabilizing the EVs; and
c) measuring expression levels of proteins selected from Gro-alpha, INF-gamma, IL-15, RANTES, SCGF-beta, and/or VEGF, using an enzyme-linked immunosorbent assay (ELISA);
wherein abnormal expression of proteins selected from Gro-alpha, INF-gamma, IL-15, RANTES, SCGF-beta, and/or VEGF compared to control, is diagnostic of autism spectrum disorder.

In some embodiments, the method comprises measuring expression levels of at least one of Gro-alpha, INF-gamma, IL-15, RANTES, SCGF-beta, and/or VEGF.

In some embodiments, the method comprises measuring expression levels of at least two of Gro-alpha, INF-gamma, IL-15, RANTES, SCGF-beta, and/or VEGF.

In some embodiments, the method comprises measuring expression levels of at least three of Gro-alpha, INF-gamma, IL-15, RANTES, SCGF-beta, and/or VEGF.

In some embodiments, the method comprises measuring expression levels of at least four of Gro-alpha, INF-gamma, IL-15, RANTES, SCGF-beta, and/or VEGF.

In some embodiments, the method comprises measuring expression levels of at least five of Gro-alpha, INF-gamma, IL-15, RANTES, SCGF-beta, and/or VEGF.

In some embodiments, the method comprises measuring expression levels of Gro-alpha, INF-gamma, IL-15, RANTES, SCGF-beta, and/or VEGF.

In some embodiments, abnormal expression of proteins selected from Gro-alpha, INF-gamma, IL-15, RANTES, SCGF-beta, and/or VEGF compared to control comprises decreased expression of Gro-alpha and/or RANTES. In some embodiments, abnormal expression of proteins selected from Gro-alpha, INF-gamma, IL-15, RANTES, SCGF-beta, and/or VEGF compared to control comprises increased expression of INF-gamma, IL-15, SCGG-beta and/or VEGF. In some embodiments, abnormal expression of proteins selected from Gro-alpha, INF-gamma, IL-15, RANTES, SCGF-beta, and/or VEGF compared to control comprises decreased expression of Gro-alpha and/or RANTES and/or increased expression of INF-gamma, IL-15, SCGG-beta and/or VEGF.

The methods described herein provide a quantitative diagnostic test for ASD. Doctors typically cannot diagnose ASD in infants or children younger than 18 months old because of their age; the tests provided herein can assist with an ASD diagnosis based on analysis of plasma samples.

### EXAMPLES

### Example 1

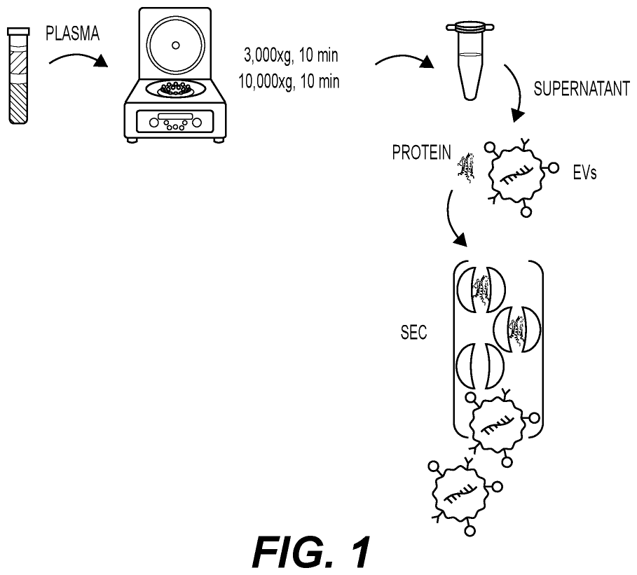

Isolation of EVs from plasma of ASD patients using size exclusion chromatography, and analysis of 1196 proteins of EV proteomes using the Olink platform with Proximity Extension Assay is conducted as shown in FIG. 1.

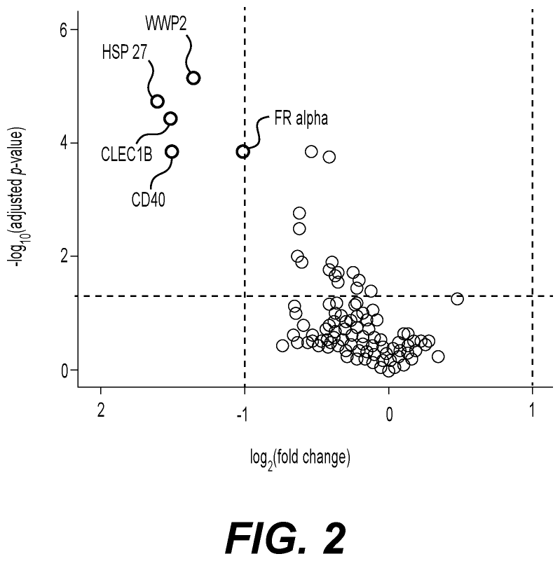

It was found that five EV proteins are downregulated in ASD: WWP2, CLEC1B, HSP-27, CD40, and FR-alpha (FIG. 2).

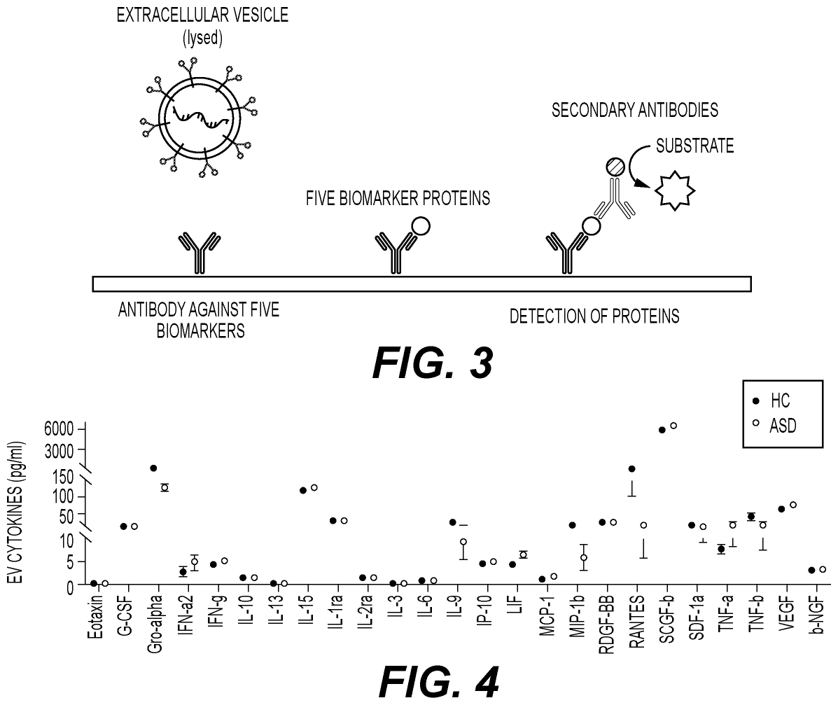

ELISA is used to detect the biomarkers (FIG. 3). The diagnostic methods described herein allow for early diagnosis of ASD where behavioral diagnoses may not be possible (e.g., when a child is young such below 18 months of age). FIG. 3 depicts an ELISA assay to detect five EV protein for ASD diagnosis. The EVs are lysed using detergent.

Our results show that a biomarker signature comprising one or more of WWP2, CLEC1B, HSP-27, CD40, and FR-alpha can be diagnostic of ASD. Our results show that profiling the EV proteins can be a useful tool for diagnosis of ASD.

### Example 2

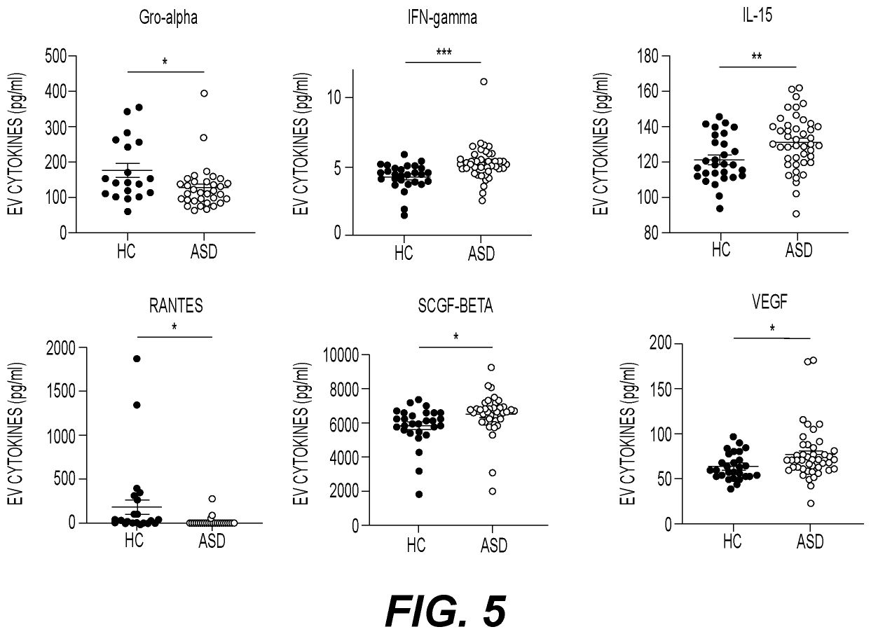

EVs from ASD plasma were isolated using size-exclusion chromatography and cytokines encapsulated in the EVs were identified by using a multiplex ELISA assay capable of detecting 48 human cytokines. We found 25 cytokines in plasma EVs (FIG. 4). Our results show six cytokines were dysregulated in EVs isolated from ASD plasma when compared to control samples. (FIG. 5). The cytokine signature for detecting ASD comprises Gro-alpha, INF-gamma, IL-15, RANTES, SCGF-beta, and/or VEGF. Our results show that profiling the EV proteins can be a useful tool for diagnosis of ASD.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for diagnosing autism spectrum disorder comprising
a) isolating extracellular vesicles (EVs) from plasma samples of subjects suffering from autism spectrum disorder; and
b) measuring protein expression levels in the extracellular vesicles.

2. A method for diagnosing autism spectrum disorder comprising:
a) isolating extracellular vesicles (EVs) from plasma samples of subjects suffering from autism spectrum disorder;
b) lysing or permeabilizing the EVs; and
c) measuring expression levels of proteins selected from WWP2, CLEC1B, HSP-27, CD40, and/or FR-alpha using an enzyme-linked immunosorbent assay (ELISA);
wherein increased expression of proteins selected from WWP2, CLEC1B, HSP-27, CD40, and/or FR-alpha compared to control is diagnostic of autism spectrum disorder.

3. The method of claim 1 or claim 2, comprising measuring expression levels of at least one of WWP2, CLEC1B, HSP-27, CD40, and/or FR-alpha.

4. The method of claim 1 or claim 2, comprising measuring expression levels of at least two of WWP2, CLEC1B, HSP-27, CD40, and/or FR-alpha.

5. The method of claim 1 or claim 2, comprising measuring expression levels of at least three of WWP2, CLEC1B, HSP-27, CD40, and/or FR-alpha.

6. The method of claim 1 or claim 2, comprising measuring expression levels of at least four of WWP2, CLEC1B, HSP-27, CD40, and/or FR-alpha.

7. The method of claim 1 or claim 2, comprising measuring expression levels of WWP2, CLEC1B, HSP-27, CD40, and FR-alpha.

8. A method for diagnosing autism spectrum disorder comprising:
a) isolating extracellular vesicles (EVs) from plasma samples of subjects suffering from autism spectrum disorder;
b) lysing or permeabilizing the EVs; and
c) measuring expression levels of proteins selected from Gro-alpha, INF-gamma, IL-15, RANTES, SCGF-beta, and/or VEGF, using an enzyme-linked immunosorbent assay (ELISA);
wherein abnormal expression of proteins selected from Gro-alpha, INF-gamma, IL-15, RANTES, SCGF-beta, and/or VEGF compared to control, is diagnostic of autism spectrum disorder.

9. The method of claim 1 or claim 8, comprising measuring expression levels of at least one of Gro-alpha, INF-gamma, IL-15, RANTES, SCGF-beta, and/or VEGF.

10. The method of claim 1 or claim 8, comprising measuring expression levels of at least two of Gro-alpha, INF-gamma, IL-15, RANTES, SCGF-beta, and/or VEGF.

11. The method of claim 1 or claim 8, comprising measuring expression levels of at least three of Gro-alpha, INF-gamma, IL-15, RANTES, SCGF-beta, and/or VEGF.

12. The method of claim 1 or claim 8, comprising measuring expression levels of at least four of Gro-alpha, INF-gamma, IL-15, RANTES, SCGF-beta, and/or VEGF.

13. The method of claim 1 or claim 8, comprising measuring expression levels of at least five of Gro-alpha, INF-gamma, IL-15, RANTES, SCGF-beta, and/or VEGF.

14. The method of claim 1 or claim 8, comprising measuring expression levels of Gro-alpha, INF-gamma, IL-15, RANTES, SCGF-beta, and/or VEGF.

15. The method of claim 8, wherein abnormal expression of proteins selected from Gro-alpha, INF-gamma, IL-15, RANTES, SCGF-beta, and/or VEGF compared to control comprises decreased expression of Gro-alpha and/or RANTES, and/or increased expression of INF-gamma, IL-15, SCGG-beta and/or VEGF.
